(19) United States
(12) Patent Application Publication
(10) Pub. No.: US 2023/0011170 A1
(43) Pub. Date: Jan. 12, 2023

(54) CARDIAC ABLATION SYSTEMS AND METHODS

(71) Applicant: EnChannel Medical Guangzhou Inc., Guangzhou (CN)

(72) Inventors: Jun Feng, Guangzhou (CN); Long Huang, Guangzhou (CN); Zhidai Mo, Guangzhou (CN); Wanting Feng, Guangzhou (CN); Long Li, Guangzhou (CN)

(73) Assignee: EnChannel Medical Guangzhou Inc., Guangzhou (CN)

(21) Appl. No.: 17/851,217

(22) Filed: Jun. 28, 2022

(30) Foreign Application Priority Data
Jul. 12, 2021 (CN) 202110787373.8

(51) Int. Cl. A61B 18/14

(52) U.S. Cl. CPC A61B 18/1492; A61B 2018/00077

(57) ABSTRACT

The present invention provides a cardiac ablation system including a spline assembly and a catheter wire. The spline assembly is provided with a plurality of electrodes and a plurality of first conductive layers encapsulated therein. A total number of the plurality of first conductive layers is corresponding to a total number of the plurality of electrodes. Each of the plurality of first conductive layers is electrically connected to each of the plurality of electrodes. The spline assembly is configured to transform into various configurations along a radial direction. A distal end of the catheter wire is connected to a proximal end of the spline assembly. The catheter wire includes a plurality of second conductive layers encapsulated therein. A total number of the plurality of second conductive layers is corresponding to the total number of the plurality of first conductive layers.

### CROSS-REFERENCES TO RELATED APPLICATIONS

The present invention claims priority to Chinese Patent Application No. 202110787373.8, entitled “BASKET-SHAPED ELECTRODE SYSTEMS”, filed on Jul. 12, 2021, which is commonly owned and incorporated by reference herein in its entirety.

### TECHNICAL FIELD

The present invention relates generally to medical devices, and more particularly to systems and methods for cardiac ablation.

### BACKGROUND

Cardiac arrhythmias can lead to heart disease and death. Atrial fibrillation (AF) is the most common persistent arrhythmia. The incidence of atrial fibrillation increases with age, and the prevalence of AF can be up to 10% in people over the age of 75 years. During AF, the atria can quiver as much as 300-600 times per minute, the heart rate is generally rapid and irregular, sometimes can be up to 100-160 beats per minute. The heart rate is not only much faster than normal but also is irregular, thereby diminishing the effective atrial contractility. AF typically increases the risks of many potentially fatal complications, including thromboembolic stroke, dilated cardiomyopathy, and congestive heart failure. Common symptoms of AF such as palpitations, chest pain, dyspnea, fatigue, and dizziness can also affect the quality of life. People with atrial fibrillation have a fivefold increase in morbidity and a twofold increase in mortality on average, compared to normal individuals.

Over the past, various types of cardiac ablation techniques have been proposed, but unfortunately, they are inadequate.

### BRIEF SUMMARY OF THE INVENTION

To overcome the above-mentioned deficiency of the prior art, the present application provides a cardiac ablation system.

To achieve the above purpose, the present application specifically discloses the following technical solutions.

According to an embodiment, the present invention provides a cardiac ablation system, which includes a spline assembly. The spline assembly includes a plurality of electrodes and a plurality of first conductive layers encapsulated therein. A total number of the plurality of first conductive layers is corresponding to a total number of the plurality of electrodes. Each of the plurality of first conductive layers is electrically connected to each of the plurality of electrodes. The spline assembly is configured to transform into various configurations along a radial direction.

The system also includes a catheter wire. A distal end of the catheter wire is connected to a proximal end of the spline assembly. The catheter wire includes a plurality of second conductive layers encapsulated therein, a total number of the plurality of second conductive layers is corresponding to the total number of the plurality of first conductive layers. Each of the plurality of first conductive layers is electrically connected to each of the plurality of second conductive layers such that each of the plurality of second conductive layers is electrically connected to each of the plurality of electrodes.

The present invention achieves various benefits. For example, the spline assembly and the catheter wire are connected. The spline assembly includes a plurality of electrodes and a plurality of first conductive layers. The catheter wire includes a plurality of second conductive layers. Each of the plurality of first conductive layers is electrically connected to each of the plurality of electrodes. Each of the plurality of first conductive layers is electrically connected to each of the plurality of second conductive layers such that each electrode on the spline assembly is independently addressable. The first conductive layers and the second conductive layers are encapsulated, thereby reducing the difficulty of organizing and soldering the wires, as well as enhancing the manufacturing efficiency and solving the problem of wire tangling in catheter lumens.

In various embodiments, the spline assembly further includes a first insulating layer. The first conductive layer is encapsulated in the first insulating layer. The first conductive layer is electrically connected to the electrode. The first conductive layer is encapsulated in the first insulating layer for isolation. Therefore, the first conductive layers may be encapsulated on the spline assembly and are isolated from each other such that the first conductive layers connected to the corresponding electrodes can work independently without affecting each other.

In various embodiments, the first insulating layer and the first conductive layer are encapsulated through injection molding. In some embodiments, the first insulating layer is printed on an outer surface of the first conductive layer through 3D printing techniques for encapsulation. Therefore, the first conductive layer can be encapsulated within the first insulating layer.

According to some embodiments, the catheter wire further includes a second insulating layer. The second conductive layer is encapsulated in the second insulating layer for isolation. When the catheter wire is connected to the spline assembly, the first conductive layer is electrically connected to the second conductive layer such that the second conductive layer is connected to the electrode. Therefore, the second insulating layers may be encapsulated on the catheter wire and are isolated from each other such that the second conductive layers connected to the corresponding first conductive layers can work independently without affecting each other.

In various embodiments, the second insulating layer and the second conductive layer are encapsulated through injection molding. In some embodiments, the second insulating layer is printed on an outer surface of the second conductive layer through 3D printing techniques for encapsulation. Therefore, the second conductive layer can be encapsulated within the second insulating layer.

In various embodiments, the spline assembly and the catheter wire are configured as an integrated structure. In some embodiments, the spline assembly and the catheter wire are connected by adhesive bonding. According to some embodiments, the spline assembly and the catheter wire may also be connected by heat fusion. Therefore, the spline assembly can be connected to the catheter wire through various means.

In some embodiments, the electrode is configured as a protrusion on a surface of the spline assembly. According to some embodiments, the electrode is configured as being lower than the surface of the spline assembly. Therefore, the electrode can generate an electrical field on the spline assembly for ablation treatment.

In some embodiments, the spline assembly further includes one or more splines configured as a cuboid structure, a fan-shaped structure, a cylindrical structure, or a hexagonal structure. The electrode is disposed on a surface of the spline assembly. The spline assembly may comprise various configurations.

In various embodiments, the system further includes an operation handle, a first catheter, a second catheter, and a third catheter. A proximal end of the catheter wire is connected to the operation handle. A proximal end of the third catheter wire is mounted on the operation handle. A distal end of the third catheter wire is connected to the proximal end of the spline assembly through a connector.

A distal end of the first catheter is connected to a distal end of the spline assembly. A proximal end of the first catheter is connected to the operation handle. The catheter wire and the first catheter are positioned within the third catheter and are configured as movable relative to the third catheter. The first catheter is controlled by the operation handle to relatively move within the third catheter such that the spline assembly may transform into various configurations. The first catheter is also configured to move within the tubular structure formed by the catheter wire and the spline assembly. Therefore, the first catheter allows for the transformation of the spline assembly through the displacement of the first catheter relative to the third catheter therein, which provides ease of operation of the system.

According to some embodiments, the operation handle includes a plug. The plug is provided with a socket. The socket is provided with a first stepped surface. A proximal end of the catheter wire is provided with a second stepped surface corresponding to the first stepped surface. When the catheter wire is connected to the operation handle, the proximal end of the catheter wire is connected to the plug, and the second conductive layer is electrically connected to the operation handle through the engagement between the first stepped surface and the second stepped surface. As such, the plug is provided with the first stepped surface, the catheter wire is provided with the second stepped surface, the catheter wire may be connected to the plug through the engagement between the first stepped surface and the second stepped surface. Such configuration is advantageous to achieve easy assembly of the catheter wire.

In various embodiments, the second stepped surface may include a plurality of second stepped elements, each of the plurality of second stepped elements is provided with a conductive layer interface such that each of the plurality of second stepped elements is corresponding to each of a plurality of conductive layer interfaces. Each of the plurality of conductive layer interfaces is connected to each second conductive layer. The first steeped surface may include a plurality of first stepped elements, each of the plurality of first stepped elements is provided with a chip interface such that each of the plurality of first stepped elements is corresponding to each of a plurality of chip interfaces. When the first stepped surface is connected to the second stepped surface, each of the plurality of conductive layer interfaces is connected to each of the plurality of chip interfaces accordingly such that the second conductive layer is electrically connected to the plug. Hence, the second conductive layer may be quickly and accurately energized when the catheter wire is connected to the plug.

According to some embodiments, the engagement between the first stepped surface and the second stepped surface may be reinforced through heat fusion to further improve the connection between the catheter wire and the plug.

In various embodiments, the third catheter includes a fourth lumen and a fifth lumen. The first catheter advances through the fourth lumen when connecting to the operation handle such that the first catheter is restricted within the third catheter. The catheter wire advances through the fifth lumen when connecting to the operation handle such that the catheter wire is restricted within the third catheter. The configuration of the third catheter enables the separation among catheters. Therefore, the configuration of the fourth lumen and the fifth lumen allows the first catheter and the catheter wire to be configured within the third catheter.

In various embodiments, the system may include a plurality of catheter wires and a plurality of fifth lumens. Each of the plurality of catheter wires advances through each of the plurality of fifth lumens, respectively, and the plurality of catheter wires may thus be separated from each other.

According to some embodiments, the system may include a plurality of catheter wires. The plurality of second conductive layers is disposed on a plurality of catheter wires accordingly. The spline assembly may include a plurality of splines. The plurality of first conductive layers is evenly disposed on the plurality of splines accordingly. The catheter wire and the plurality of splines may be configured as a tubular structure, thus allowing the plurality of catheter wires to be connected to the plurality of splines.

In some embodiments, the first conductive layers of at least two splines are connected to the second conductive layers of a single catheter wire. A total number of the first conductive layers of the at least two splines equals a number of the second conductive layers of the single catheter wire. Therefore, the first conductive layers of two or more splines may be connected to a single catheter wire, which is advantageous to reduce the total number of the catheter wires and provides easy installation.

It is to be appreciated that embodiments of the present invention provide many advantages over conventional techniques. The third catheter forms a conduction block which allows for the separation between wires and catheters such that the problem of wire tangling can be readily avoided, and the assembly efficiency can be vastly improved.

The present invention achieves these benefits and others in the context of known technology. However, a further understanding of the nature and advantages of the present invention may be realized by reference to the latter portions of the specification and attached drawings.

### BRIEF DESCRIPTION OF THE DRAWINGS

The following diagrams are merely examples, which should not unduly limit the scope of the claims herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this process and scope of the appended claims.

FIG. 1 is a perspective view of a cardiac ablation system configured in a tubular profile, in accordance with various embodiments.

FIG. 2 is an enlarged view of region A of FIG. 1, in accordance with various embodiments.

FIG. 3 is a perspective view of a cardiac ablation system configured in an expanded profile (e.g., spindle-shaped), in accordance with various embodiments.

FIG. 4 is an enlarged view of region B of FIG. 3, in accordance with various embodiments.

FIG. 5 is a schematic view of a cardiac ablation system wherein a second catheter comprising a spline assembly and a catheter wire is not configured as a tubular structure, in accordance with a first embodiment.

FIG. 6 is a schematic view of a cardiac ablation system wherein a spline assembly and a catheter wire are configured as a tubular structure, in accordance with the first embodiment.

FIG. 7 is an enlarged view of region C of FIG. 6, in accordance with various embodiments.

FIG. 8 is a sectional view of a cardiac ablation system, in accordance with the first embodiment.

FIG. 9 is a sectional view of a cardiac ablation system, in accordance with the first embodiment.

FIG. 10 is a schematic view illustrating a connection between a catheter wire and a socket, in accordance with the first embodiment.

FIG. 11 is a sectional view of an operation handle, in accordance with the first embodiment.

FIG. 12 is a sectional view of a third catheter, in accordance with the first embodiment.

FIG. 13 is a perspective view of a connector, in accordance with the first embodiment.

FIG. 14 is a sectional view of a third catheter, in accordance with a second embodiment.

FIG. 15 is a schematic view of a cardiac ablation system wherein a spline assembly and a catheter wire are not configured as a tubular structure, in accordance with a third embodiment.

FIG. 16 is a sectional view of a cardiac ablation system, in accordance with the third embodiment.

FIG. 17 is a flow diagram illustrating a method for manufacturing a spline assembly, in accordance with various embodiments.

According to the aforementioned figures, below is a list of reference symbols: 1—spline assembly; 11—first conductive layer; 12—first insulating layer; 2—catheter wire; 21—second conductive layer; 22—second insulating layer; 23—a second stepped surface; 3—electrode; 4—operation handle; 41—operation knob; 42—translation knob; 43—chip; 431—socket; 432—first stepped surface; 5—first catheter; 51—mounting cap; 6—third catheter; 61—first lumen; 62—second lumen; 63—third lumen; 64—fourth lumen; 65—fifth lumen; 7—connector; 71—groove; 81—second catheter.

### DETAILED DESCRIPTION OF THE INVENTION

The present invention is directed to cardiac ablation apparatuses. A specific embodiment provides a cardiac ablation system, which includes a spline assembly. The spline assembly includes a plurality of electrodes and a plurality of first conductive layers encapsulated therein. The spline assembly is connected to a catheter wire configured for electrical connection and signal transmission. The catheter advances through a third catheter and is connected to an operation handle, which is configured to manipulate the positioning and transformation of the spline assembly. The spline assembly may transform into various configurations for ablation treatment.

To treat cardiac arrhythmias, ablation can be performed using an ablation catheter to cause changes in the tissues. The purpose of ablation is to destroy the tissues related to underlying cardiac arrhythmias and to create transmural and durable lesions. For ablation, catheters can be designed into various configurations, including a flexible design that allows the catheter to be inserted as a compacted shaft which can subsequently expand into a spindle-shaped arrangement. Such flexible configuration can expand after entering the endocardial space and can later be folded upon the completion of ablation before exiting the endocardial space. Catheters are provided with electrodes, which can physically engage with the cardiac wall and perform ablation thereon.

However, traditional catheter manufacturing techniques involve welding wires to electrodes. When the number of electrodes increases, the number of wires increases accordingly, making it more difficult to organize and solder the cables, as well as causing the problem of wire tangling in the catheter lumen.

To make the objectives, technical solutions, and advantages of the present disclosure clearer and easier to understand, the present disclosure will be further described in detail below through embodiments in conjunction with the accompanying drawings. It should be appreciated that the specific embodiments described here are merely utilized to explain the present disclosure, rather than limiting the present disclosure.

The serial numbers assigned to the components herein, such as “first,” “second,” “third,” and such are merely utilized for illustration purposes. Unless otherwise specified or indicated, the term “a plurality of” means two or more. The terms “connection” and “fixed” mentioned in the disclosure shall be given a broad interpretation, for example, “connection” may be understood as a fixed connection, a removable connection, an integrated connection, or an electrical connection, which includes direct and/or indirect connection via a medium. The following description is presented to enable one of ordinary skill in the art to make and use the invention and to incorporate it in the context of particular applications.

In the description of the present disclosure, it should be appreciated that the orientations or position relationships indicated by the terms “upper,” “lower,” and such are based on the orientations or position relationships shown in the drawings, and shall not be understood as a limitation to the present disclosure. In addition, based on the context, it is also to be appreciated that a first element being configured “on” or “under” a second element may be that the first element is directly positioned “on” or “under” the second element, and/or the first element is indirectly positioned “on” or “under” the second element through a medium.

The following description is presented to enable one of ordinary skill in the art to make and use the invention and to incorporate it in the context of particular applications. Various modifications, as well as a variety of uses in different applications, will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification, and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the Claims herein is not intended to invoke the provisions of 35 U.S.C. 112, Paragraph 6.

Please note, if used, the labels left, right, front, back, top, bottom, forward, reverse, clockwise, and counter-clockwise have been used for convenience purposes only and are not intended to imply any particular fixed direction. Instead, they are used to reflect relative locations and/or directions between various portions of an object.

### First Embodiment

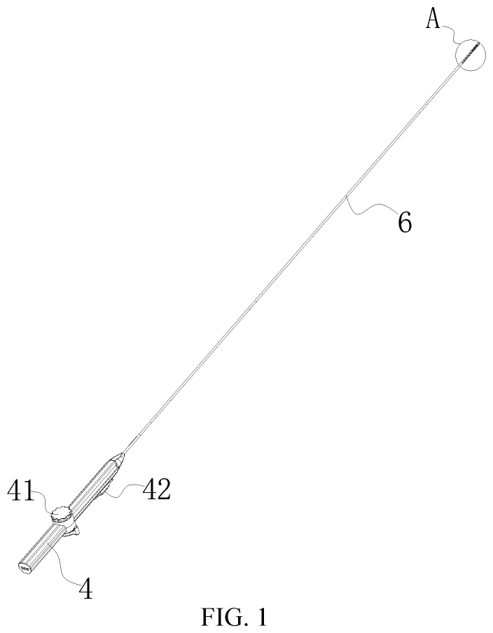

FIG. 1 is a perspective view of a cardiac ablation system configured in a tubular profile, in accordance with various embodiments. This diagram is merely an example, which should not unduly limit the scope of the claims. One of ordinary skill in the art would recognize many variations, alternatives, and modifications.

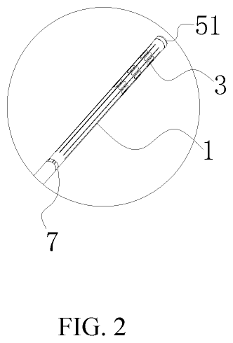

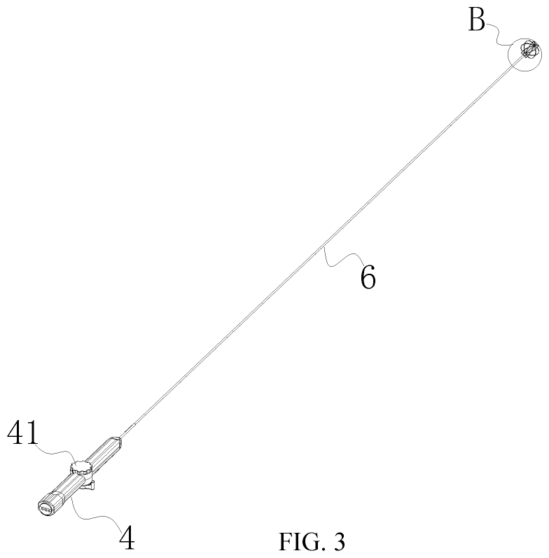

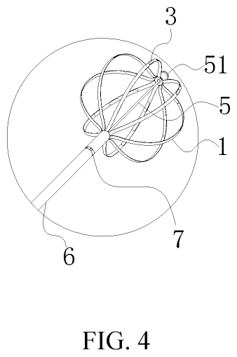

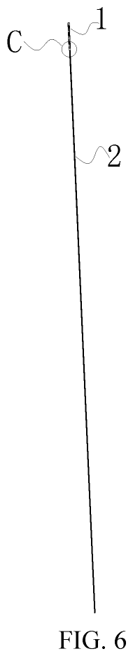

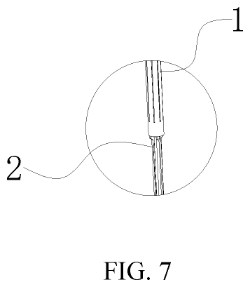

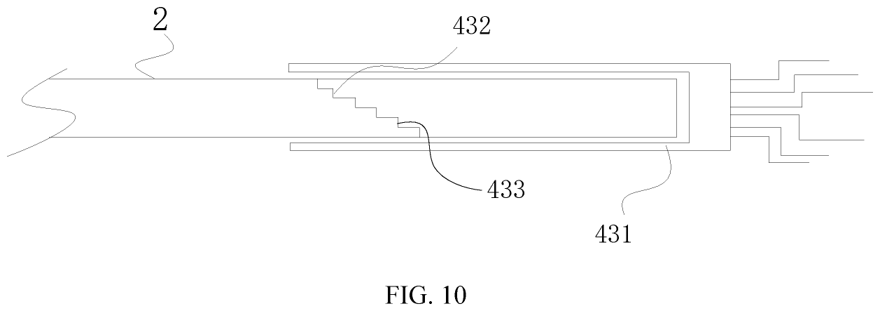

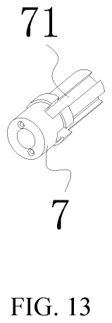

Now referring back to FIGS. 1-13. In some embodiments, a cardiac ablation system includes a spline assembly 1 and a catheter wire 2. Spline assembly 1 includes a plurality of splines. The plurality of splines defining a hollow region within the spline assembly 1. Spline assembly is configured to transform into various configurations including a first configuration and a second configuration. For example, the first configuration may be configured as a tubular structure. The second configuration may be configured as a spindle-shaped structure where the plurality of splines expands outwardly. Spline assembly 1 is provided with a plurality of electrodes 3. The plurality of electrodes 3 may be positioned on the plurality of splines. The plurality of electrodes 3 is configured to be in contact with and deliver ablation energy to a tissue designated for ablation and receive an intracardiac electrocardiogram (IECG) signal. A plurality of first conductive layers 11 corresponding to the plurality of electrodes 3 are encapsulated in spline assembly 1. Each first conductive layer 11 is electrically connected to a corresponding electrode 3, which is configured for electric discharge, thus allowing spline assembly 1 to be deployed for tissue ablation. Electrode 3 is further configured to be in contact with an atrial wall to receive IECG signals and deliver ablation energy for ablation treatment. Spline assembly 1 is configured for translation along a radial direction to transform into a spindle-shaped configuration, which is advantageous for abutting an atrial wall to conduct ablation treatment after entering the endocardial space. A distal end of catheter wire 2 is coupled to a proximal end of spline assembly 1. Catheter wire 2 is configured to record the ECG signal and transmit ablation energy to the tissue designated for ablation. A plurality of second conductive layers 21 are encapsulated in catheter wire 2, and the number of the plurality of second conductive layers 21 correspond to the number of the plurality of first conductive layers 11. Each of the plurality of first conductive layers 11 is electrically connected to each of the plurality of second conductive layers 21 such that each of the plurality of second conductive layers 21 is electrically connected to each of the plurality of electrodes 3. Catheter wire 2 is configured to record IECG signals and deliver ablation energy to the plurality of electrodes 3 provided on spline assembly 1, thus allowing each electrode to be independently addressable. In some embodiments, the system comprises a plurality of catheter wires 2. The plurality of catheter wires 2 and spline assembly 1 may be configured as a tubular structure, thus allowing spline assembly 1 to transform into an expanded (e.g., spindle-shaped) configuration.

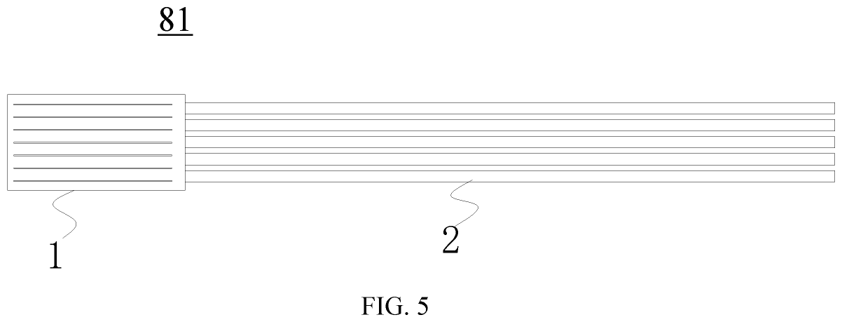

According to some embodiments, a cardiac ablation system further includes an operation handle 4, a first catheter 5, a second catheter 81, and a third catheter 6. As shown in FIG. 5, spline assembly 1 and catheter wire 2 together form second catheter 81. That is, second catheter 81 comprises spline assembly 1 and catheter wire 2. During the use of spline assembly 1 and catheter wire 2, a proximal end of catheter wire 2 is coupled to and positioned within operation handle 4, a proximal end of third catheter 6 is mounted on a distal end of the operation handle 4, a distal end of third catheter 6 is connected to a proximal end of spline assembly 1 through a connector 7. The first catheter 5 may be positioned within the hollow region defined by the plurality of splines of spline assembly 1. A distal end of first catheter 5 is connected to a distal end of spline assembly 1, and a proximal end of first catheter 5 is connected to and positioned within operation handle 4. Catheter wire 2 and first catheter 5 are both disposed within third catheter 6, and catheter wire 2 and first catheter 5 can both move relative to third catheter 6. First catheter 5 can move relative to and within third catheter 6 through the manipulation of operation handle 4 to allow for the transformation of spline assembly 1. First catheter 5 can move within a tubular structure formed by catheter wire 2 and spline assembly 1. First catheter 5 is configured to drive a transformation of the spline assembly 1 through the displacement/movement of the first catheter 5. The relative movement of first catheter 5 within third catheter 6 allows for the transformation of spline assembly 1, and thus enhancing maneuverability of spline assembly 1. Operation handle 4 is configured to configure the displacement of first catheter 5 to drive the transformation of the spline assembly 1.

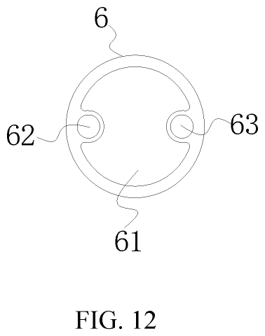

Specifically, in some embodiments, third catheter 6 has a length of 110 cm. The proximal end of third catheter 6 may be applied with AB glue and later be inserted into operation handle 4 from a distal end of operation handle 4. Third catheter 6 then can be firmly fixed to operation handle 4 once the AB glue hardens. According to some embodiments, third catheter 6 is provided with three lumens therein, including a first lumen 61, a second lumen 62, and a third lumen 63. First catheter 5 and catheter wire 2 are both positioned within first lumen 61, catheter wire 2 and first catheter 5 are not completely fixed within first lumen 61 of third catheter 6. Such configuration merely restricts the movement of catheter wire 2, allowing first catheter 5 and catheter wire 2 to be configured as relatively movable within third catheter 6. For example, first catheter 5 advances through the first lumen 61 of third catheter 6 and extends from a distal end of first catheter 5 to a distal end of the spline assembly 1. Catheter wire 2 advances through first lumen 61 of third catheter 6, a distal end of catheter wire 2 is connected to a proximal end of the spline assembly 1. Second lumen 62 and third lumen 63 are positioned opposite to each other within third catheter 6. Both second lumen 62 and third lumen 63 are provided with a bending mechanism (e.g., a pull wire, not shown) for configuring a movement of third catheter 6. A first end of the bending mechanism is coupled to the distal end of third catheter 6, and a second end of the bending mechanism is coupled to and positioned within operation handle 4. A user may manipulation operation handle 4 to configure the bending mechanism to selectively bend, steer, deploy, deflect, rotate and/or modify the shape of third catheter 6, and thus enhance the flexibility of the catheter. As shown in FIG. 12, in some embodiments, a first diameter of first lumen 61 is configured as greater than a second diameter of second lumen 62 and a third diameter of third lumen 63, making it possible to accommodate both first catheter 5 and catheter wire 2 within first lumen 61. As such, first catheter 5 and catheter wire 2 are relatively movable within first lumen 61. In some embodiments, when a plurality of catheter wires 2 are configured as a tubular structure and form a hollow channel therein, first catheter 5 may be moveable within the hollow channel formed by the plurality of catheter wires 2.

According to some embodiments, the bending mechanism is coupled to a connector 7. A distal end of the bending mechanism is provided with a spherical structure. Connector 7 is provided with a mounting hole, the mounting hole is configured to create a mounting space to mount the spherical structure therein for movement restriction, thereby coupling the bending mechanism to connector 7. A diameter of a first end of connector 7 is configured as smaller than a diameter of third catheter 6. A diameter of a second end of connector 7 is configured as smaller than a diameter of the tubular structure formed by spline assembly 1. The first end of connector 7 is coupled to the distal end of third catheter 6. Through the manipulation of the bending mechanism, connector 7 may be firmly attached to the distal end of third catheter 6. The second end of connector 7 is coupled to the proximal end of spline assembly 1. When first catheter 5 is connected to spline assembly 1, by pulling spline assembly 1, spline assembly 1 can be firmly attached to the second end of connector 7. Therefore, the first end of connector 7 is connected to third catheter 6 and the second end of connector 7 is connected to spline assembly 1. Third catheter 6 is thus connected to spline assembly 1 through connector 7.

In some embodiments, catheter wire 2 may bend at its distal end which is coupled to spline assembly 1 such that a diameter of catheter wire 2 is configured as smaller than the diameter of spline assembly 1 when catheter wire 2 and spline assembly 1 are configured as the tubular structure. Such configuration also enables catheter wire 2 to enter third catheter 6 and makes it easier to connect third catheter 6 to spline assembly 1 and advance the catheters into the endocardial space for ablation. Connector 7 is provided with a groove 71. When third catheter 6 is connected to spline assembly 1 through connector 7, a bending area of catheter wire 2 may be positioned at groove 71. Catheter wire 2 may extend from groove 71 and enter third catheter 6. Catheter wire 2 is configured as relatively moveable within third catheter 6. Groove 71 creates space for the bending area of catheter wire 2 to release stress during bending, which is advantageous to reduce operating stress and enhance the steerability and maneuverability of the system.

In some embodiments, the distal end of first catheter 5 is provided with a mounting cap 51. Mounting cap 51 may be integrated with first catheter 5 or glued to first catheter 5. Mounting cap 51 is mounted on the distal end of spline assembly 1, and thus connecting first catheter 5 to the distal end of spline assembly 1.

In some embodiments, spline assembly 1 further includes a first insulating layer 12. First conductive layer 11 is encapsulated within first insulating layer 12 and first conductive layer 11 is connected to electrode 3. First conductive layer 11 being encapsulated within first insulating layer 12 provides isolation among the plurality of first conductive layers 11. First conductive layer 11 may therefore be encapsulated within spline assembly 1 and each first conductive layer 11 is isolated from each other such that each first conductive layer 11 connected to the corresponding electrode 3 can operate independently. In some embodiments, first insulating layer 12 may be printed on an outer surface of first conductive layer 11 through 3D printing techniques to encapsulate first conductive layer 11. First conductive layer 11 can therefore be encapsulated within first insulating layer 12. In some embodiments, when manufacturing the spline assembly 1 using 3D printing techniques, a first base layer (not shown) initially configured in a substantially planar form may serve as a foundation for one or more layers to be laid. Various materials and thickness configurations may be employed to achieve desirable mechanical and process characteristics. For example, the various layers (including the first base layer) may be configured with different/same thickness to achieve desirable mechanical properties (e.g., elastic modulus, tensile strength, elongation, hardness, fatigue limit, etc.). In some embodiments, the first base layer may be configured as thicker than the other layers to obtain desired elastic modulus and hardness such that durability and repeatable high accuracy can be achieved. For example, the first base layer may have a width of 1 mm and a thickness of 30 μm to 50 μm. It is to be appreciated that the design of the catheter system including the choice of material is critical to the steerability, torque, and flexibility of the catheter system to achieve desirable performance. For example, the first base layer comprises at least one of a Polyethylene terephthalate (PET) material, a Polyimide (PI) material, a Polyurethane (PU) material, combinations thereof, and the like.

The first base layer may at least be partially coated with a first functional layer (e.g., first insulating layer 12) configured for electrical connection. For example, each first functional layer may have a thickness of 15 μm to 25 μm. According to some embodiments, first conductive layer 11 has high ductibility and comprises at least one of a gold material, a silver material, and/or a copper material, combinations thereof, and the like. The first functional layer may further be plated with an insulating layer (e.g., first insulating layer 12) configured for isolation between the first functional layers. First insulating layer 12 has great flexibility and includes at least one of an Epoxy material, a polyimide material, a polyurethane material, a fused wire material, a fused deposition modeling (FDM) ceramic material, a wood-plastic composite material, and/or a FDM support material, combinations thereof, and the like. Insulating layers with higher flexibility may improve the durability of spline assembly 1. In various embodiments, first insulating layer 12 may be printed on first conductive layer 11 in a layer-by-layer manner to encapsulate first conductive layer 11. Once the printing process is completed, the planar first base layer may be wound around a longitudinal axis to form a cylindrical form. It is to be appreciated that the planar first base layer may be slit to form individual splines separated from each other. Prior to advancing spline assembly 1 into the endocardial space, a pre-deformation of the splines (e.g., configured in a slightly curved form) is advantageous to improve the microstructure and the mechanical properties of spline assembly 1.

According to some embodiments, catheter wire 2 may further include a second insulating layer 22. Second conductive layer 21 is encapsulated by second insulating layer 22 such that each second conductive layer 21 is isolated from each other. When catheter wire 2 is connected to spline assembly 1, first conductive layer 11 is electrically connected to second conductive layer 21 so that second conductive layer 21 is electrically connected to electrode 3. Second conductive layer 21 may be encapsulated within catheter wire 2 and each second conductive layer 21 is isolated from each other such that each second conductive layer 21 connected to the corresponding first conductive layer 11 can also operate independently. In some embodiments, second insulating layer 22 may be printed on an outer surface of second conductive layer 21 through 3D printing techniques to encapsulate second conductive layer 21. Second conductive layer 21 can therefore be encapsulated within second insulating layer 22. In some embodiments, when manufacturing catheter wire 2 using 3D printing techniques, a second base layer (not shown) initially configured in a planar form may serve as a foundation for one or more layers to be laid. Various materials and thickness configurations may be employed to achieve desirable mechanical and process characteristics. For example, the various layers (including the second base layer) may be configured with different/same thickness to achieve desirable mechanical properties (e.g., elastic modulus, tensile strength, elongation, hardness, fatigue limit, etc.). In some embodiments, the second base layer may be configured as thicker than the other layers to obtain desired elastic modulus and hardness such that durability and repeatable high accuracy can be achieved. For example, the second base layer may have a width of 1 mm and a thickness of 30 μm to 50 μm. It is to be appreciated that the design of the catheter system including the choice of material is critical to the steerability, torque, and flexibility of the catheter system to achieve desirable performance. For example, the second base layer comprises at least one of a Polyethylene terephthalate (PET) material, Polyimide (PI) material, Polyurethane (PU) material, combinations thereof, and the like.

The second base layer may at least be partially coated with a second functional layer (e.g., second insulating layer 22) configured for electrical connection. For example, each second functional layer may have a thickness of 15 μm to 25 μm. According to some embodiments, second conductive layer 21 comprises at least one of a gold material, a silver material, and/or a copper material, combinations thereof, and the like. The second functional layer may further be plated with a second insulating layer (e.g., second insulating layer 22) configured for isolation between the second functional layers. Second insulating layer 22 includes at least one of an Epoxy material, a polyimide material, a polyurethane material, a fused wire material, a fused deposition modeling (FDM) ceramic material, a wood-plastic composite material, and/or a FDM support material, combinations thereof, and the like. Insulating layers with higher flexibility may improve the durability of catheter wire 2. In various embodiments, second insulating layer 22 may be printed on second conductive layer 21 in a layer-by-layer manner to encapsulate second conductive layer 21.

According to some embodiments, spline assembly 1 is integrated with catheter wire 2 to form a connection between spline assembly 1 and catheter wire 2. In some embodiments, spline assembly 1 and catheter wire 2 may be integrally printed through 3D printing techniques to form an integrated structure of spline assembly 1 and catheter wire 2, which may be later configured into a compact profile (e.g., a tubular structure).

In some embodiments, electrode 3 is configured as a protrusion disposed on a surface of spline assembly 1. Electrode 3 is configured to generate an electric field for ablation treatment.

In some embodiments, the system comprises a plurality of catheter wires 2. A plurality of second conductive layer 21 is evenly disposed on the plurality of catheter wires 2. Spline assembly 1 includes a plurality of splines. A plurality of first conductive layers 11 are evenly disposed on the plurality of splines. The plurality of catheter wires may be connected to the plurality of splines. The plurality of catheter wires 2 and the plurality of splines 1 may together be configured as a tubular structure.

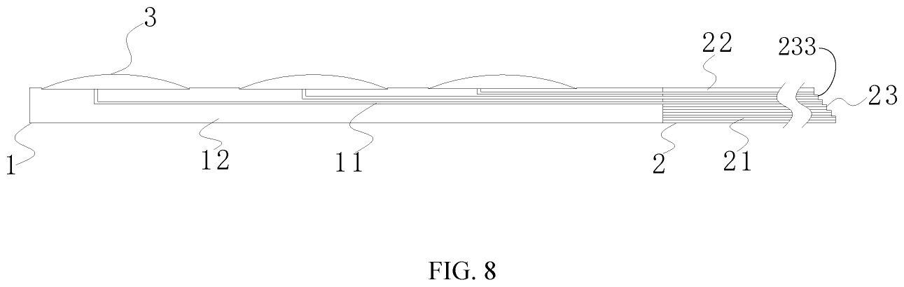

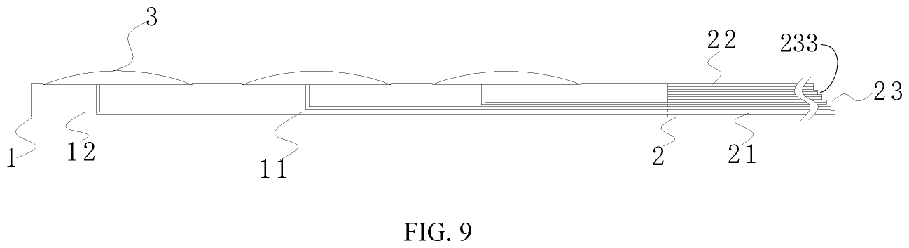

In some embodiments, the first conductive layers 11 of at least two splines are connected to the second conductive layers 21 of a single catheter wire 2. A total number of the first conductive layers 11 of the at least two splines equals a number of the second conductive layers 21 of the single catheter wire 2. Therefore, the first conductive layers 11 of two or more splines may be connected to a single catheter wire 2, which is advantageous to reduce the total number of the catheter wires 2 and facilitate easy installation. In various embodiments, spline assembly 1 includes a plurality of splines configured as a cuboid structure, a fan-shaped structure, a cylindrical structure, and/or a hexagonal structure, and the like. The plurality of splines is arranged along a longitudinal axis of spline assembly 1. Spline assembly may comprise various configurations. Electrode 3 is positioned on a surface of the spline. In some embodiments, the proximal end of spline assembly 1 is connected to four catheter wires 2, which may be advanced into first lumen 61 of third catheter 6 when spline assembly 1 is transformed into the tubular structure. In some embodiments, spline assembly 1 is provided with 24 electrodes 3 positioned thereon. Accordingly, a total number of corresponding first conductive layers is 24, each catheter wire 2 includes six layers of second conductive layer 21 encapsulated therein. According to some embodiments, electrode 3 may be configured as a protrusion with a height of 0.05-0.50 mm. For example, electrode 3 is configured as a protrusion with a height of 0.2 mm positioned on the surface of spline assembly 1. It is to be appreciated that the distribution and deployment of electrodes 3 on spline assembly may be flexibly designed in a manner that facilitates contact between electrodes 3 and the atrial wall. For example, electrode 3 is positioned close to the distal end of spline assembly 1. According to some embodiments, spline assembly 1 comprises eight splines, each of which is provided with three electrodes 3 and three layers of first conductive layer 11, such that every two splines may share a single catheter wire 2. FIG. 8 is a sectional view of a single spline where electrode 3, first conductive layer 11, and second conductive layer 21 all comprise gold materials and are configured in a stacked configuration printed in a layer-by-layer manner. FIG. 9 is a sectional view of a pair of splines where second conductive layers 21 of the two splines are separated by an insulation material (e.g., first insulating layer 12 and second insulating layer 22) along a vertical direction.

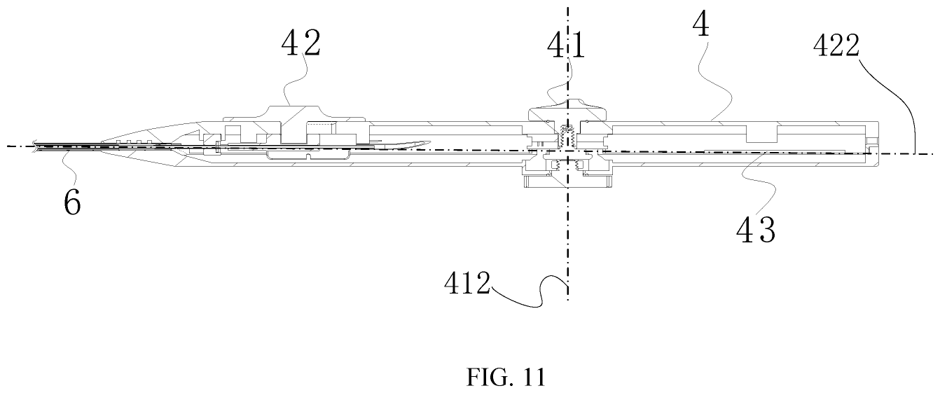

As shown in FIG. 11, in some embodiments, when the proximal end of third catheter 6 is connected to the distal end of operation handle 4, first catheter 5, the bending mechanism, and catheter wire 2 that positioned within third catheter 6 may all extend from the proximal end of third catheter 6 and be connected to an inside of operation handle 4. Operation handle 4 includes an operation knob 41, a translation knob 42, and a chip 43. Operation knob 41 is positioned on a side of a housing of operation handle 4 along a radial direction. Operation knob 41 defines a second longitudinal axis 412 perpendicular to a first longitudinal axis 422 defined by operation handle 4 therethrough. Operation knob 41 may rotate about the second longitudinal axis 412. The bending mechanism is connected to operation knob 41. The bending mechanism may be manipulated through the rotation of operation knob 41 to deflect third catheter 6. Catheter wire 2 may extend from third catheter 6 and connect to chip 43 of operation handle 4. Chip 43 is configured to control electrode 3 which is connected to catheter wire 2 such that IECG signals recorded by electrode 3 can be transmitted to a peripheral device and/or ablation energy can be delivered to electrode 3 of spline assembly 1. In some embodiments, first catheter 5 extends from third catheter 6 and is connected to translation knob 42 within operation handle 4. Translation knob 42 permits the movement of first catheter 5 within third catheter 6, thereby allowing for the transformation of spline assembly 1 that is coupled to first catheter 5 for ablation therapy.

In some embodiments, chip 43 is provided with a plug that comprises a socket 431. Socket 431 includes a first stepped surface 432 comprising one or more first stepped elements 433. The proximal end of catheter wire 2 is provided with a second stepped surface 23 comprising one or more second stepped elements 233 configured to engage with first stepped surface 432. When catheter wire 2 is connected to operation handle 4, the proximal end of catheter wire 2 may be connected to the plug. Second conductive layer 21 of catheter wire 2 may be electronically connected to operation handle 4 through the engagement between first stepped surface 432 and second stepped surface 23. The plug is provided with first stepped surface 432 and catheter wire 2 is provided with second stepped surface 23. Catheter wire 2 may engage with the plug through the engagement between first stepped surface 432 and second stepped surface 23 to achieve fast installation of catheter wire 2. The engagement between second stepped surface 23 and chip 43 disposed within operation handle 4 allows for easy assembly of the catheter system comprising multiple electrodes 3.

In some embodiments, each second stepped element 233 of the second stepped surface 23 is provided with a conductive layer interface (not shown) such that each conductive layer interface is connected to each second conductive layer 21. Each first stepped element 422 of first stepped surface 432 is provided with a chip interface (not shown). When first stepped surface 432 is connected to second stepped surface 23, each conductive layer interface is connected to each chip interface accordingly such that second conductive layer 21 is electrically connected to the plug of chip 43. Each second conductive layer 21 may work independently without affecting each other. Such configuration provides fast installation and accurate electrical connections. Once catheter wire 2 is advanced into the plug of chip 43, connection and sealing quality may be enhanced by adhesive application methods or heat fusion methods. Chip 43 of operation handle 4 is configured to transmit signals to the wire leads, which may be welded to an electrical plug, thereby establishing electrical connections between electrode 3 and a peripheral device. According to some embodiments, first stepped surface 432 of the plug is provided with six chip interfaces and second stepped surface 23 of catheter wire 2 is provided with six conductive layer interfaces correspondingly.

The working mechanism of the cardiac ablation system: during the use of cardiac ablation system of the present invention, the distal end of spline assembly 1 is advanced into the patient's body to contact with the tissue designated for ablation. A user may operate translation knob 42 of operation handle 4 to manipulate first catheter 5 to transform spline assembly 1 from a compact profile (e.g., a tubular configuration) to an expanded profile (e.g., a spindle-shaped configuration). The transformation of spline assembly 1 improves the contact between the ablation device and the atrial wall. Chip 43 is configured to transmit IECG signals to the peripheral device and/or to deliver ablation energy to electrodes 3 disposed on spline assembly 1 to perform ablation therapy. Upon the completion of ablation, a user may operate translation knob 42 to configure first catheter 5 to restore spline assembly 1 to the compact profile (e.g., the tubular configuration) for removal. During the ablation process, when spline assembly 1 needs to be deflected at various angles for ablation, by operating operation knob 41 to configure the bending mechanism to manipulate third catheter 6, the flexible positioning of spline assembly 1 can thus be realized for effective ablation therapy.

### Second Embodiment

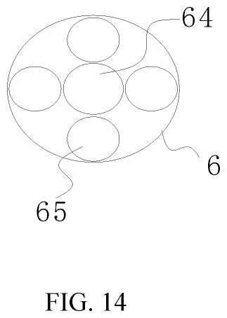

Now referring to FIG. 14. A cardiac ablation system according to the present disclosure is provided. The difference between the second embodiment and the aforementioned first embodiment lies in that third catheter 6 includes a fourth lumen 64 and a fifth lumen 65 instead of first lumen 61, second lumen 62, and third lumen 63. When first catheter 5 is connected to operation handle 4, first catheter 5 advances through fourth lumen 64 such that first catheter 5 may be restricted within third catheter 6 and can move within third catheter 6. When catheter wire 2 is connected to operation handle 4, catheter wire 2 advances through fifth lumen 65 such that catheter wire 2 may be restricted within third catheter 6 and can move within third catheter 6. When the system includes a plurality of catheter wires 2 and a plurality of fifth lumens 65. Each fifth lumen 5 is configured to allow a catheter wire 2 to advance therethrough. Third catheter 6 is configured to separate first catheter 5 and catheter wire 2. Catheter wire 2 may be configured as movable within third catheter 6. Fourth lumen 64 and fifth lumen 65 allow for the positioning of first catheter 5 and catheter wire 2 within third catheter 6. First catheter 5 and catheter wire 2 are movable relative to and within third catheter 6.

In some embodiments, third catheter 6 has a length of 110 cm. A proximal end of third catheter 6 may be applied with AB glue and later be inserted into operation handle 4 from a distal end of operation handle 4. Third catheter 6 then can be firmly fixed to operation handle 4 once the AB glue hardens. According to some embodiments, third catheter 6 includes fourth lumen 64 and fifth lumen 65. When third catheter 6 is connected to the distal end of spline assembly 1, first catheter 5 is positioned within fourth lumen 64. First catheter 5 is not completely fixed within fourth lumen 64 of third catheter 6. Such configuration merely restricts the movement of first catheter 5, allowing first catheter 5 to be configured as movable relative to and within third catheter 6. For example, first catheter 5 advances through fourth lumen 64 of the third catheter 6. Catheter wire 2 advances through fifth lumen 65 of the third catheter 6 such that first catheter 5 is separated from catheter wire 2.

In some embodiments, when the proximal end of spline assembly 1 is coupled to four catheter wires 2, each of the four catheter wires 2 is configured as advancing through each of four fifth lumens 65, respectively. Spline assembly 1 and catheter wire 2 together form second catheter 81. That is, second catheter 81 comprises spline assembly 1 and catheter wire 2. Catheter wire 2 is not completely fixed within fifth lumen 65 of third catheter 6. Such configuration merely restricts the movement of catheter wire 2, allowing catheter wire 2 to be configured as movable relative to and within third catheter 6. It is to be appreciated that separate lumens (e.g., fourth lumen 64 and fifth lumen 65) provide separate individual channels for wires and catheters to advance therethrough, which is advantageous to avoid entanglement among wires and catheters.

In some embodiments, third catheter 6 does not include a bending mechanism.

Further details of spline assembly 1, catheter wires 2, and operation handle 4 may be similar to those described in the first embodiment and are not described in detail hereafter.

### Third Embodiment

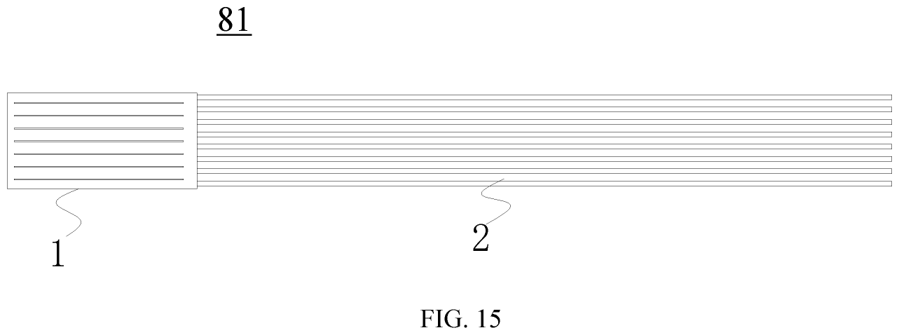

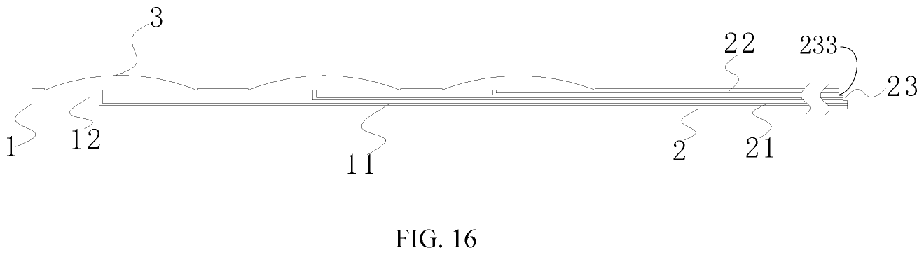

Now referring to FIGS. 15 and 16. A cardiac ablation system according to the present disclosure is provided. The difference between the third embodiment and the aforementioned first and second embodiments is that spline assembly 1 includes one or more splines configured as a cuboid structure, a fan-shaped structure, a cylindrical structure, or a hexagonal structure, and the like. The plurality of splines is arranged along a longitudinal axis of spline assembly 1. As shown in FIG. 15, spline assembly 1 and catheter wire 2 together form second catheter 81. That is, second catheter 81 comprises spline assembly 1 and catheter wire 2. Spline assembly 1 may comprise various configurations. For example, electrode 3 is disposed on a surface of the spline. In some embodiments, a proximal end of spline assembly 1 is connected to eight catheter wires 2. Spline assembly 1 includes eight splines. In some embodiments, spline assembly 1 is provided with twenty-four electrodes 3 positioned thereon. Accordingly, a total number of corresponding first conductive layers is twenty-four. Each spline is provided with three electrodes 3 such that each catheter wire 2 includes three layers of second conductive layer 21 encapsulated therein. Electrode 3 is configured as a protrusion with a height of 0.2 mm positioned on a surface of spline assembly 1. Electrode 3 is positioned close to the distal end of spline assembly 1. According to some embodiments, spline assembly 1 comprises eight splines, each of which is provided with three electrodes 3 and three layers of first conductive layer 11. The proximal end of spline assembly 1 is connected to eight catheter wires 2 such that each spline of spline assembly 1 is connected to a catheter wire 2, respectively. FIG. 16 is a sectional view of a single spline where electrode 3, first conductive layer 11, and second conductive layer 21 all include gold materials and are configured in a stacked configuration printed layer by layer with insulating materials (e.g., first insulating layer 12 and second insulating layer 22) positioned therebetween for separation.

In some embodiments, third catheter 6 has a length of 110 cm. A proximal end of third catheter 6 may be applied with AB glue and later be inserted into operation handle 4 from a distal end of operation handle 4. Third catheter 6 then can be firmly fixed to operation handle 4 once the AB glue hardens. According to some embodiments, third catheter 6 includes a fourth lumen 64 and eight fifth lumens 65. When third catheter 6 is connected to the distal end of spline assembly 1, first catheter 5 is positioned within fourth lumen 64. First catheter 5 is not completely fixed within fourth lumen 64 of third catheter 6. Such configuration merely restricts the movement of first catheter 5, allowing first catheter 5 to be configured as movable relative to and within third catheter 6. In some embodiments, when the proximal end of spline assembly 1 is connected to eight catheter wires 2, each of the eight catheter wires 2 is configured as advancing through each of eight fifth lumens 65, respectively. Catheter wire 2 is not completely fixed within fifth lumen 65 of third catheter 6. Such configuration merely restricts the movement of catheter wire 2, allowing catheter wire 2 to be configured as movable relative to and within third catheter 6. It is to be appreciated that separate lumens (e.g., fourth lumen 64 and fifth lumen 65) provide separate individual channels for wires and catheters to advance therethrough, which is advantageous to avoid entanglement among wires and catheters.

In some embodiments, third catheter 6 does not include a bending mechanism.

Further details of spline assembly 1, catheter wires 2, and operation handle 4 may be similar to those described in the first embodiment and are not described in detail hereafter.

In some embodiments, first insulating layer 12 and first conductive layer 11 may be encapsulated through injection molding methods. Second insulating layer 22 and second conductive layer 21 may be encapsulated through injection molding methods.

In various embodiments, electrode 3 may be configured as being lower than the surface of spline assembly 1. According to some embodiments, electrode 3 may be configured as being lower than the surface of spline assembly 1 by 0.1 mm.

In some embodiments, spline assembly 1 and catheter wire 2 may be connected through adhesive bonding methods, heat fusion methods, and the like.

In some embodiments, the distal end of first catheter 5 may be connected to a guidewire (not shown), which extends out of first catheter 5. The guidewire is configured for safety protection, direction guiding, and advancement and positioning improvement. The guidewire is movable within first catheter 5.

It is to be appreciated that embodiments of the present invention provide many advantages over conventional techniques. The connection between spline assembly 1 and catheter wire 2 allows for enhanced encapsulation of first conductive layer 11 provided on the spline assembly as well as second conductive layer 21 provided on catheter wire 2, thus reducing wire clutter and allowing for easy welding. Additionally, wire encapsulation techniques described herein can achieve a high level of manufacturing efficiency while eliminating the problem of wire tangling in the catheter lumen.

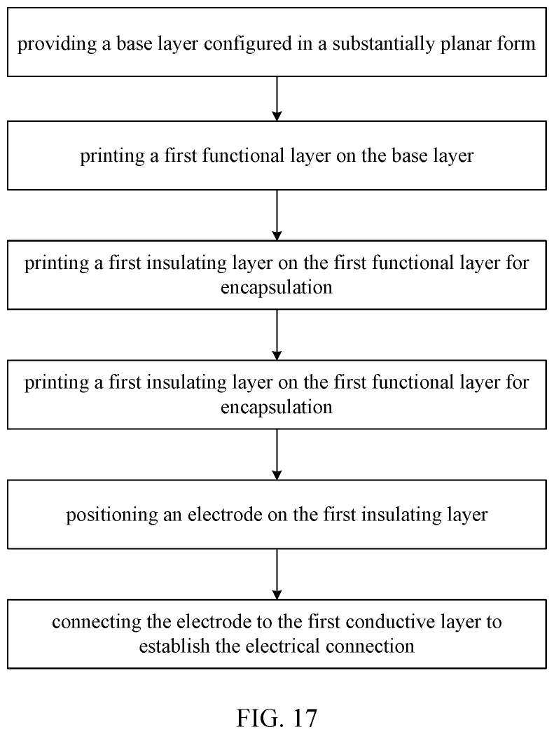

FIG. 17 is a flow diagram illustrating a method for manufacturing a spline assembly. A method for manufacturing a spline assembly comprises:

S1, providing a base layer configured in a substantially planar form, the base layer comprising at least one of a Polyethylene terephthalate (PET) material, a Polyimide (PI) material, a Polyurethane (PU) material;

S2, printing a first functional layer on the base layer, the first functional layer being configured for establishing an electrical connection, the first functional layer comprising at least one of a gold material, a silver material, and a copper material;

S3, printing a first insulating layer on the first functional layer for encapsulation, the first insulating layer comprises at least one of an Epoxy material, a polyimide material, a polyurethane material, a fused wire material, a fused deposition modeling (FDM) ceramic material, a wood-plastic composite material, and a FDM support material;

S4, positioning/printing an electrode on the first insulating layer, the electrode being configured as a protrusion provided on an outer surface of the first insulating layer, the electrode is electrically connected to the first conductive layer to establish the electrical connection.

In some embodiments, the base layer has a width of 1 mm and a first thickness of 30 μm to 50 μm. The first functional layer has a second thickness of 15 μm to 25 μm. The electrode has a height of 0.05 mm to 0.50 mm. For example, the electrode has a height of 0.2 mm.

What is described above are only several implementations of the present application, and these embodiments are specific and detailed, but not intended to limit the scope of the present application. It should be understood by the skilled in the art that various modifications and improvements can be made without departing from the conception of the present application, and all fall within the protection scope of the present application. Therefore, the patent protection scope of the present application is defined by the appended claims.

What is claimed is:

1. A cardiac ablation system, comprising:
a spline assembly comprising a plurality of splines, the plurality of splines defining a hollow region within the spline assembly, the spline assembly being configured to transform into a first configuration and a second configuration;
a plurality of electrodes positioned on the plurality of splines, the plurality of electrodes being configured to be in contact with and deliver ablation energy to a tissue designated for ablation and receive an IECG signal;
a first catheter positioned within the hollow region, the first catheter being configured to drive a transformation of the spline assembly through a displacement of the first catheter;
a catheter wire configured to record the IECG signal and transmit ablation energy to the tissue designated for ablation;
a second catheter comprising the spline assembly and the catheter wire; and
a third catheter defining a first lumen, a second lumen, and a third lumen therethrough;
wherein the first catheter advances through the first lumen of the third catheter and extends from a distal end of the first catheter to a distal end of the spline assembly;
wherein the catheter wire advances through the first lumen of the third catheter, a distal end of the catheter wire is connected to a proximal end of the spline assembly;
wherein the first catheter and the catheter wire are configured as movable within the third catheter.

2. The system of claim 1, wherein the first configuration of the spline assembly is configured as a tubular structure.

3. The system of claim 1, wherein the second configuration of the spline assembly is configured as a spindle-shaped structure.

4. The system of claim 1, wherein the spline assembly further comprises a plurality of first conductive layers and a plurality of first insulating layers, each of the plurality of first conductive layers is electrically connected to each of the plurality of electrodes;
wherein each of the plurality of first conductive layers is encapsulated within in each of the plurality of first insulating layers.

5. The system of claim 4, wherein the catheter wire further comprises a plurality of second conductive layers and a plurality of second insulating layers, each of the plurality of second conductive layers is electrically connected to each of the plurality of first conductive layers, and each of the plurality of second conductive layers is encapsulated within in each of the plurality of second insulating layers.

6. The system of claim 5, wherein a single catheter wire is electrically connected to one or more splines through a connection between the plurality of first conductive layers and the plurality of second conductive layers thereof.

7. The system of claim 1, further comprising:
an operation handle for configuring the displacement of the first catheter to drive the transformation of the spline assembly, the operation handle comprises a first stepped surface including a plurality of first stepped elements.

8. The system of claim 7, wherein the catheter wire further comprises a second stepped surface comprising a plurality of second stepped elements configured to engage with the first stepped surface of the operation handle such that the catheter wire is electrically connected to the operation handle.

9. The system of claim 1, wherein the second lumen is provided with a bending mechanism for configuring a movement of the third catheter.

10. The system of claim 1, wherein the catheter wire is connected to the spline assembly through a connector, the connector comprises a groove configured for the catheter wire to advance therethrough.

11. The system of claim 1, wherein a first diameter of the first lumen is configured as greater than a second diameter of the second lumen and/or a third diameter of the third lumen.

12. A cardiac ablation system, comprising:
a spline assembly comprising a spline, the spline assembly being configured to transform into a first configuration and a second configuration;
an electrode positioned on the spline of the spline assembly, the electrode being configured to be in contact with and deliver ablation energy to a tissue designated for ablation and receive an IECG signal;
a first catheter positioned within a hollow region defined by the spline assembly, the first catheter being configured to drive a transformation of the spline assembly between the first configuration and the second configuration through a displacement of the first catheter;
a catheter wire configured to record the IECG signal and transmit ablation energy to the tissue designated for ablation;
a second catheter comprising the spline assembly and the catheter wire; and
a third catheter defining a fourth lumen and a fifth lumen therethrough;
wherein the first catheter advances through the fourth lumen of the third catheter and the catheter wire advances through the fifth lumen of the third catheter such that the first catheter is separated from the catheter wire;
wherein the first catheter and the catheter wire are configured as movable within the third catheter.

13. The system of claim 12, wherein the spline further comprises a first conductive layer and a first insulating layer, the first conductive layer is configured to be electrically connected to the electrode;
wherein the first conductive layer is at least partially plated with the first insulating layer for encapsulation.

14. The system of claim 13, wherein the first conductive layer comprises at least one of a gold material, a silver material, and a copper material.

15. The system of claim 13, wherein the first insulating layer comprises at least one of an Epoxy material, a polyimide material, a polyurethane material, a fused wire material, a fused deposition modeling (FDM) ceramic material, a wood-plastic composite material, and a FDM support material.

16. The system of claim 12, wherein the spline is configured as a cuboid structure, a fan-shaped structure, a cylindrical structure, or a hexagonal structure.

17. A method for manufacturing a spline assembly, comprising:
providing a base layer configured in a substantially planar form, the base layer comprising at least one of a Polyethylene terephthalate (PET) material, a Polyimide (PI) material, a Polyurethane (PU) material;
printing a first functional layer on the base layer, the first functional layer being configured for establishing an electrical connection, the first functional layer comprising at least one of a gold material, a silver material, and a copper material;
printing a first insulating layer on the first functional layer for encapsulation, the first insulating layer comprises at least one of an Epoxy material, a polyimide material, a polyurethane material, a fused wire material, a fused deposition modeling (FDM) ceramic material, a wood-plastic composite material, and a FDM support material; and
positioning/printing an electrode on the first insulating layer, the electrode being configured as a protrusion provided on an outer surface of the first insulating layer, the electrode being electrically connected to the first conductive layer to establish the electrical connection.

18. The method of claim 17, wherein the base layer has a width of 1 mm and a first thickness of 30 μm to 50 μm.

19. The method of claim 17, wherein the first functional layer has a second thickness of 15 μm to 25 μm.

20. The method of claim 17, wherein the electrode is configured as a protrusion with a height of 0.2 mm.
